(19) United States
(12) Patent Application Publication
(10) Pub. No.: US 2004/0097568 A1
(43) Pub. Date: May 20, 2004

(54) Crystalline form of losartan potassium

(75) Inventors: Manne Satyanarayana Reddy, Hyderabad (IN); Sajja Eswaraiah, Hyderabad (IN); Ravinder Reddy Koppera, Hyderabad (IN); Vajrala Venkata Reddy, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/629,316

(22) Filed: Jul. 29, 2003

(30) Foreign Application Priority Data
Jul. 29, 2002 (IN) 568/MAS/2002

(51) Int. Cl. A61K 31/4178; C 07D 4 3/02

(52) U.S. Cl. 514/381; 548/254

(57) ABSTRACT

A compound that is a crystalline Form III of losartan potassium is provided. Also provided are compositions containing the compound and methods for its preparation.

### CROSS REFERENCE TO RELATED APPLICATIONS

This application claims benefit of a filing date of an Indian Patent Application No. 568/MAS/2002, filed Jul. 29, 2002, the contents of which are expressly incorporated herein by reference.

### BACKGROUND OF THE INVENTION

The enzyme renin acts on angiotensinogen, or alpha-2 globulin, to produce angiotensin I, which is converted by angiotensin converting-enzyme to angiotensin II (AII), a hormone known to be a strong receptor-mediated vasopressor acting on target mammalian cells. Losartan potassium (2-butyl-4-chloro-1-[[2′-(1H-tetrazol-5-yl)[1,1′-biphenyl]-4-yl]methyl]-1H-imidazole-5-methanol, potassium salt) inhibits the action of angiotensin II at the receptor site and is useful, therefore, in alleviating AII-associated hypertension. The administration of losartan potassium to a mammal afflicted with AII-associated atherosclerosis, high cholesterol or hypertension reduces blood pressure and total cholesterol. Losartan potassium may be administered either as a step-wise combined therapy (diuretic first), or in the same formulation with a diuretic, such as furosemide, or hydrochlorothiazide, for an enhanced anti-hypertensive effect while treating atherosclerosis and reducing cholesterol levels. Administration of a compound of this invention with a non-steroidal anti-inflammatory drug (NSAID) can prevent renal failure associated with the administration of the latter.

U.S. Pat. No. 5,138,069 discloses and claims losartan, its derivatives and pharmaceutically acceptable salts, including the potassium salt, as well as compositions and a method of treatment using pharmaceutically acceptable salts of losartan and its derivatives. The prior patent also discloses a process for the preparation of losartan and its derivatives, which comprises de-protecting trityl losartan with 3.4 N hydrochloric acid to free the losartan base, and then adding aqueous potassium hydroxide-isopropanol solution to convert the free base to its potassium salt. There is a continuing need for new forms of losartan potassium and/or new methods of their preparation.

### SUMMARY OF THE INVENTION

In accordance with one aspect, the invention provides a crystalline Form III of losartan potassium. The crystalline Form III of losartan potassium is a high melting point solid that contains residual solvents within permissible limits under ICH guidelines. This crystalline form of losartan potassium is well suited for use in pharmaceutical and veterinary formulations, and is active as an angiotension II (AII) blocker, and effective for the treatment of hypertension and congestive heart failure, among other diseases and conditions.

In accordance with another aspect, the invention also provides a composition containing losartan potassium as a solid, wherein at least 80% by weight of the solid losartan potassium is its crystalline Form III. Various embodiments of this aspect of the invention are also provided.

In accordance with yet another aspect, the invention provides a pharmaceutical or veterinary composition containing crystalline Form III of losartan potassium and a pharmaceutically or veterinarily acceptable carrier or diluent. Various embodiments of this aspect of the invention are also provided.

In accordance with yet another aspect, the invention provides a process for preparing crystalline Form III of losartan potassium by a) providing a potassium salt of losartan as a solution in a first alcoholic solvent; b) cooling said solution thereby causing separation of a solid mass; and c) isolating said solid mass which is the crystalline Form III of losartan potassium. In one embodiment of this aspect of the invention, the step of providing the starting potassium salt of losartan includes reacting the precursor trityl losartan with a potassium base. In another embodiment, the providing step includes dissolving crystalline Form I of potassium losartan in a mixture of an alcohol and an aromatic solvent. Various embodiments of this aspect of the invention are also provided.

### DESCRIPTION OF DRAWINGS

FIG. 1 shows the X-ray powder diffraction of crystalline Form III of losartan potassium.

FIG. 2 is differential scanning colorimetry thermogram of crystalline Form III of losartan potassium.

FIG. 3 is an infrared spectrum of crystalline Form III of losartan potassium.

### DESCRIPTION OF PREFERRED EMBODIMENTS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below.

The crystalline compound designated herein as “crystalline Form III”, and referred to hereinafter as crystalline Form III of losartan potassium, is a new crystalline polymorph of losartan potassium different from known polymorphs. It is characterized via X-ray powder diffraction, DSC and/or infrared spectroscopy.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes but is not limited to a solution, a suspension, a gel, an ointment, an emulsion and/or mixtures thereof. The term composition is intended to encompass a product comprising the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the crystalline Form III of losartan potassium, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

“Therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “substantially free of” in reference to a composition, as used herein, means that the substance from which the composition is free of cannot be detected by methods known to those skilled in the art.

Losartan potassium has the chemical structure

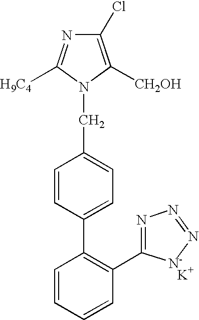

Polymorphic Forms I and II of losartan potassium are known in the art. These forms, as well as their preparation, were disclosed in U.S. Pat. No. 5,608,075, incorporated herein by reference in its entirety, and specifically for the purposes of showing how Forms I and II are prepared and characterized. Form I of losartan potassium was prepared as a white solid by treating losartan base with a potassium hydroxide solution, adding the resulting mass to a refluxing azeotropic mixture of a cyclohexane and isopropanol solvent, and continuing the distillation of solvent until the moisture content reaches <0.05%. Form II of losartan potassium was obtained by heating Form I of losartan potassium in an open vessel at a temperature of 255° C. The polymorphic Forms I and II of losartan potassium were identified and differentiated by X-ray diffraction, differential scanning calorimetry (DSC) and infrared spectroscopy.

A new crystalline form of losartan potassium have now been discovered. The invention provides such new crystalline form of losartan potassium and the process for preparing the new crystalline form of losartan potassium. In particular, the inventors found that a new polymorph of losartan potassium, referred to herein as Form III, may be obtained by crystallizing/precipitating losartan potassium, as a solid, from a solvent containing an alcohol. In one preferred embodiment, crystalline Form III of losartan potassium is obtained by cooling a solution of losartan potassium in an alcoholic solvent to separate a solid mass and then filtering the solid mass, which is the product. In one variant of this embodiment, the starting solution of losartan potassium is obtained by de-protecting trityl losartan with potassium hydroxide. In another variant, the starting solution of losartan potassium is obtained by dissolving the Form I of losartan potassium in an alcoholic solvent. The preferred alcoholic solvents include lower alcohols and/or mixtures of lower alcohols with aromatic solvents. The processes of the embodiments of the invention are described below in greater details.

The crystalline Form III of losartan potassium obtained as described herein may be characterized by several analytical methods. X-ray diffraction provides a convenient and practical means for qualitative and quantitative characterization of crystalline powders.

The qualitative identification of a form of a compound from its X-ray powder diffraction pattern is based upon the position of the lines (in terms of 2 theta) and their relative intensities. The diffraction angle 2 theta is determined by the spacing between a particular set of planes. Using the Bragg equation, the distance d is readily calculated from the known wavelength of the source and the measured angle. Identification of the crystalline form is empirical. By measuring the intensity of the diffraction lines and comparing them with standards, it is possible to make a quantitative analysis of crystalline mixtures. Qualitative information can be converted to quantitative data by measuring the peak heights. Two methods that are used to analyze X-ray diffraction quantitatively are the Internal Standard Method and the External Standard Method. The Internal Standard Method is the preferred procedure for analyzing powdered systems. This method measures a known quantity of a reference powder which is added to an unknown powder. The mass absorption coefficient of the mixture need not be known in advance. Any number of constituents in the mixture may be quantified independently, including the amorphous (non-crystalline) components. The External Standard Method is used to analyze solid systems when the mass absorption co-efficient is known. It allows the quantification of one or more components in a system, which may contain an amorphous fraction.

The X-ray diffraction pattern for the crystalline form exhibits a diffraction pattern with a unique set of diffraction peaks that can be expressed in 2 theta angles, d-spacing values and relative peak intensities. 2 Theta diffraction angles and corresponding d-spacing values account for positions of various peaks in the X-ray powder diffraction pattern. D-spacing values are calculated with observed 2 theta angles and copper K(α1) wavelength using the Bragg equation.

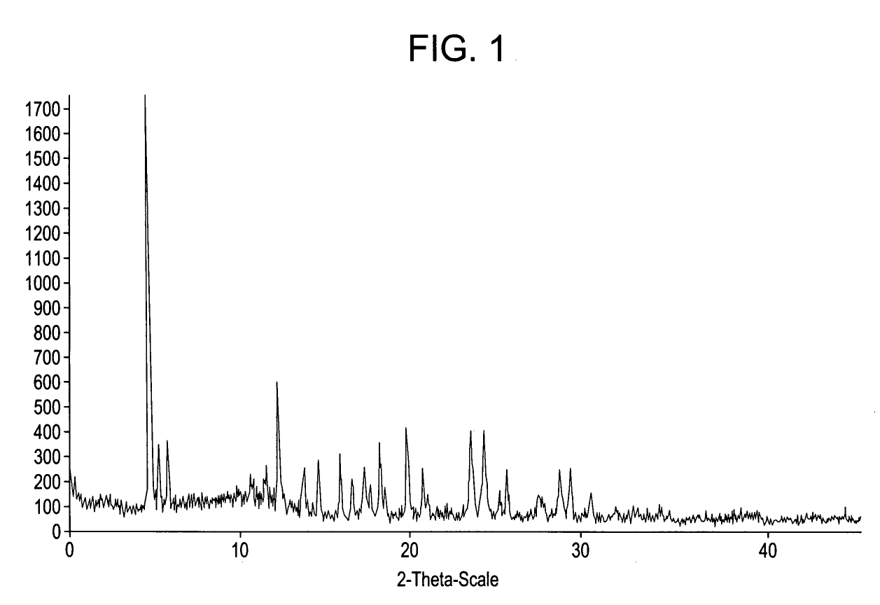

FIG. 1 shows an X-ray powder diffractogram of the crystalline Form III of losartan potassium obtained by the inventors. The X-ray powder diffraction pattern of the FIG. 1 was measured on a Bruker Axs, D8 Advance Powder X-ray Diffractometer with Cu K alpha-1 Radiation source. The characteristic X-ray powder diffraction pattern for crystalline Form III of losartan potassium, as expressed in terms of the angle 2θ (Degrees), and percentage Intensity (% I), is shown below:

| 2-θ | Intensity |
|---|---|
| (°) | (I/Io) |
| 7.154 | 100 |
| 13.911 | 29.9 |
| 20.728 | 23.4 |
| 24.904 | 22.0 |
| 24.192 | 21.1 |
| 19.293 | 16.1 |
| 8.042 | 14.6 |
| 7.583 | 14.4 |
| 16.043 | 13.6 |
| 17.194 | 13.3 |
| 28.908 | 12.5 |
| 29.474 | 11.9 |
| 26.088 | 11.5 |
| 21.576 | 11.5 |
| 15.267 | 11.1 |
| 18.483 | 11.0 |
| 17.794 | 10.1 |
| 13.233 | 7.60 |
| 19.571 | 7.20 |
| 30.614 | 6.80 |

However, some margin of error is not avoidable in X-ray powder diffraction measurements. Slight variations in observed 2 theta angles or d-spacing values are expected based on the specific diffractometer employed the analyst and the sample preparation technique. More variation is expected for the relative peak intensities. Identification of the crystal form of a compound should be based primarily on observed 2 theta angles with lesser importance attributed to relative peak intensities. Thus, the assigned margin of error in the 2 theta angles for Form III of losartan potassium is approximately ±0.09 for each of the peak assignments. In view of the assigned margin of error, the crystalline Form III of losartan potassium may be characterized by an X-ray powder diffraction pattern that includes five or more peaks selected from the group consisting of peaks with 2 theta angles of 0.15±0.09, 7.58±0.09, 8.04±0.09, 12.38±0.09, 13.23±0.09, 13.91±0.09, 15.27±0.09, 16.04±0.09, 17.19±0.09, 17.79±0.09, 18.48±0.09, 18.76±0.09, 19.29±0.09, 19.57±0.09, 20.73±0.09, 21.58±0.09, 24.19±0.09, 24.90±0.09, 25.67±0.09, 26.09±0.09, 27.77±0.09, 28.91±0.09, 29.47±0.09 and 30.61±0.09.

The invention also provides a composition containing losartan potassium as a solid in which at least 80%, by total weight of losartan potassium in the composition, is its crystalline Form III. The remainder of losartan potassium in the composition, i.e., 20% or less of the total weight of losartan potassium may be, for example, the crystalline forms I and II of losartan potassium. In a more preferred embodiment, the composition contains at least 90% of the crystalline Form III form with respect to total weight of losartan potassium in the composition. Yet more preferably, the composition contains at least 95% of the crystalline Form III form with respect to total weight of losartan potassium in the composition. In the most preferred embodiment, the composition is substantially free of the crystalline forms I and II of losartan potassium. In one preferred variant, the composition includes at least a small amount of crystalline Form I or II losartan potassium, preferably, crystalline losartan potassium. In a non-limiting example, the composition includes at least 80% of crystalline Form III of losartan potassium and at least 1% crystalline Form III of losartan potassium. In another non-limiting example, the composition includes at least 80% of crystalline Form III of losartan potassium and at least 5% crystalline Form III of losartan potassium.

The relative amounts of crystalline Forms of losartan potassium may be determined, for example, by X-ray diffraction, which is adaptable to quantitative applications because the intensities of the diffraction peaks of a given compound in a mixture are proportional to the faction of the material in the mixture. Thus, the relative amounts of given crystalline forms of losartan potassium may be characterized by X-ray diffraction.

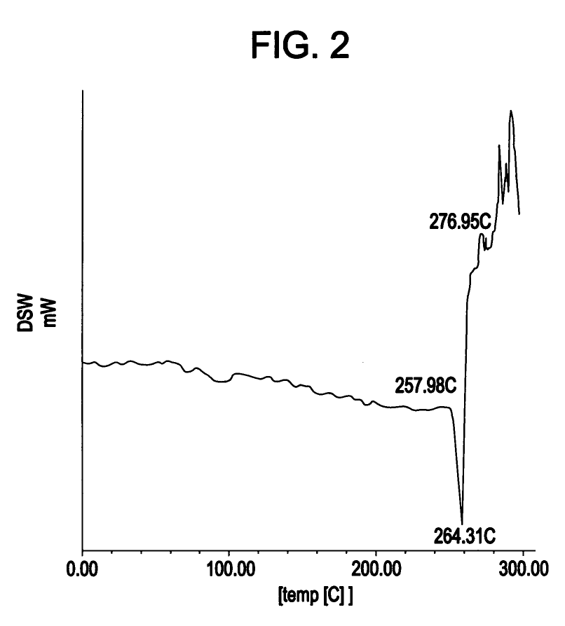

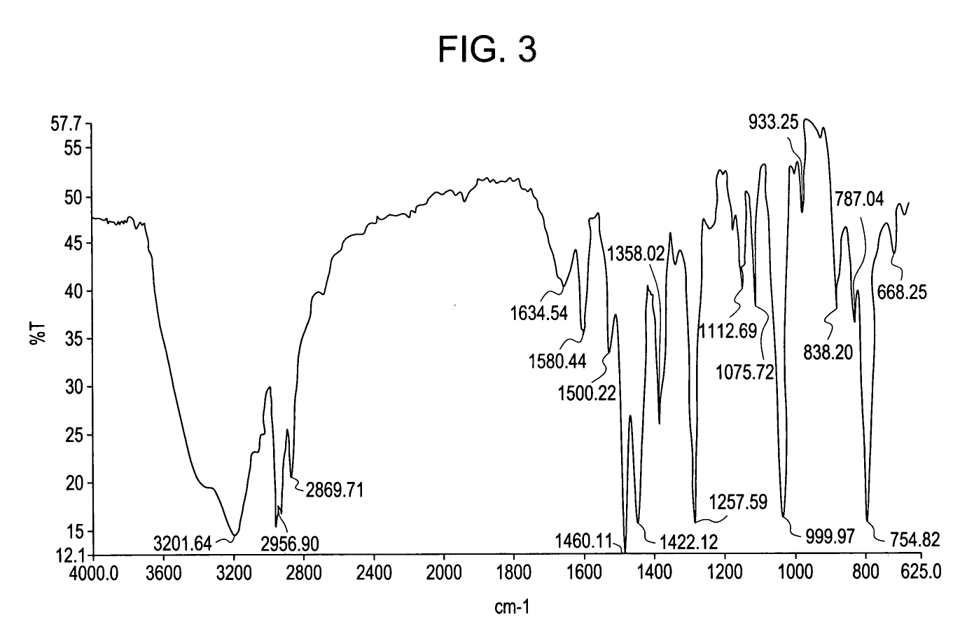

The crystalline Form III of losartan potassium also may be characterized by Differential Scanning Calorimetry (DSC) and Infrared spectroscopy. The DSC thermogram of the crystalline Form III of losartan potassium is shown in FIG. 2. The DSC thermogram was obtained on Schimadzu differential scanning calorimeter operated within a temperature range of 50-250° C., and a heating rate of 5° C./minute. It may be seen that the crystalline Form III compound's DSC thermogram exhibits a significant endo peak at about 264° C. The crystalline Form III also has a characteristic infrared (IR) spectrum shown in FIG. 3. IR spectral data were obtained on a Perkin-Elmer FT-IR instrument by the KBr transmission method. IR spectrum shows identifiable significant peaks at about 1580, 1460, 1422, 1358, 1257, 1112, 1075, 999, 754, and 668 cm−1. The crystalline Form III of losartan potassium melts over a range of temperatures of 254-260° C. as determined by the capillary method.

The crystalline Form III of losartan potassium described herein may be used as an active ingredient in pharmaceutical formulations. Thus, the invention also provides a pharmaceutical or veterinary composition that includes the crystalline Form III of losartan potassium as the active ingredient, and a pharmaceutically or veterinarily acceptable carrier or diluent. Suitable pharmaceutically acceptable excipients include starches, sugars, celluloses, such as microcrystalline cellulose, hydroxypropyl cellulose, and hydroxypropylmethyl cellulose, diluents, granulating agents, lubricants, binders, disintegrating agents, and the like. Generally, the pharmaceutical compositions of the invention are prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

The active ingredient may be present in the composition in an amount of from about 0.1 to about 99.9% by weight of the composition; in a second range, from about 0.5 to 99% by weight of the composition; in a third range, from about 1 to about 90% by weight of the composition; in a forth range, from about 2 to about 20% by weight of the composition. The composition may be solid or liquid, and may be provided as a powder, tablets, dragees, capsules, oil, cream, solution, emulsion, suspension, or as a solid or liquid spray. The composition is also provided in the form of a topical or systemic formulation. Preferred are oral, injectable, transdermal, implantable, inhalable, transmucosal, and dermal formulations, and in particular solid oral formulations that may be in the form of an enteric coated and/or a delayed release formulation. The carrier or diluent may be solid or liquid, and preferably solid and in the form of a cellulosic material, a starch, a polyhydroxylated alcohol, derivatives thereof, or mixtures thereof. Another preferred group of carriers and diluents are derivatized cellulosic materials, starches, polyhydroxylated alcohols, mixtures thereof, and mixtures thereof with underivatized carriers or diluents such as cellulosic materials, starches such as potato and maize starches, polyhydroxylated alcohols, and mixtures thereof or with other known carriers and diluents. Other carriers are also contemplated and are not excluded for use with this composition. The composition may also contain other ingredients known in the art for use in veterinary and pharmaceutical compositions. Examples are lubricants, disintegrants, coloring agents, anti-hygroscopic agents, binders, pH adjusting agents, flavoring agents, and aromatic agents. However, others are also contemplated for use herein.

The more preferred oral solid preparation is a tablet. A tablet may be prepared by direct compression, wet granulation, or molding, of the crystalline losartan potassium with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine, a mixture of the powdered compound moistened with an inert liquid diluent are suitable in the case of oral solid dosage forms (e.g., powders, capsules, and tablets). If desired, tablets may be coated by standard techniques. The crystalline losartan potassium described herein may be formulated into typical disintegrating tablet, or into a controlled or extended release dosage forms. Examples of suitable controlled release formulation vehicles are disclosed in U.S. Pat. Nos. 3,845,770; 3,916,899; 3,536,809; 3,598,123; and 4,008,719, the disclosures of which are hereby incorporated by reference in their entirety.

The crystalline Form III of losartan potassium may be prepared as generally described above and exemplified in Examples 1, 2 and 3 herein. Either a non-mixed lower alcohol or a mixture of lower alcohol with an aromatic solvent is used for crystallization/precipitation. Non-limiting examples of preferred alcohols include methanol, ethanol, isopropanol, n-butanol, iso-butanol, tert-butanol, and mixtures thereof. Methanol is preferred. Non-limiting example of suitable aromatic solvents include benzene, xylene, toluene, ethyl benzene, or their mixtures. The preferred aromatic solvent is toluene. If used, de-protection of trityl losartan is accomplished in an aqueous potassium base, such as potassium hydroxide, preferably on reflux in methanol. The preferred molar ratio between trityl losartan and potassium hydroxide ranges from 0.5:1.5 to 1.5:0.5. The amount of solvent prior to cooling crystallization is selected to allow a substantial portion of dissolved losartan potassium to separate from solution. Thus, it may be necessary to distill a portion of the solvent prior to cooling. Once crystalline Form III of losartan potassium separates from solution, it may be filtered and dried in the usual manner, typically at from about 30 to about 100° C. The resulting crystalline Form III of losartan potassium has been characterized as a crystalline, non-solvated, high melting temperature, free flowing solid that is well suited for use in pharmaceutical applications.

In one preferred variant, the process of obtaining Form III of losartan potassium includes:

a) refluxing the reaction solution of 2-n-Butyl-4-chloro-1-[(2′-(1-triphenylmethyl-1H-tetrazole-5-yl)-1,1′-biphenyl-4-yl)methyl]1H-imidazole-5-methanol(trityl Losartan) in a mixture of aqueous solution of potassium hydroxide and C₁-C₄ straight or branched chain alcoholic solvents such as methanol, ethanol, isopropanol, n-butanol, iso-butanol and tertiary butanol, preferably methanol;

b) distilling off the solvent from the reaction solution of step (a);

c) cooling the reaction mass of step (b) to a temperature of 25-40° C. accompanied by addition of water;

d) filtering the reaction mass of step (c);

e) washing the filtrate obtained in step (d) with water followed by aromatic solvents such as benzene, xylene, toluene or ethyl benzene, preferably toluene;

f) separating the layers from reaction solution of step (e) and accompanied by distilling the water from aqueous layer;

g) azeotropic distillation of water traces from the reaction mass of step (f) using water immisible aromatic solvents such as benzene, xylene, toluene or ethyl benzene, preferably toluene;

h) dissolving the compound of step (g) in C₁-C₄ straight or branched chain alcoholic solvents such as methanol, ethanol, isopropanol, n-butanol, iso-butanol and tertiary butanol, preferably methanol;

i) optionally subjecting the reaction solution of step (h) with carbon;

j) distilling off the solvent from reaction solution of step (i) accompanied by cooling the resulting reaction mass to a temperature of 0-50° C.;

k) filtering the compound obtained in step (j) followed by drying the compound at temperature of 30-100° C. to afford the crystalline Form III of losartan potassium.

In one preferred variant, the process of obtaining Form III of losartan potassium includes:

a. heating the crystalline Form I of losartan potassium salt in water immisible aromatic solvents such as benzene, xylene, toluene or ethyl benzene, preferably toluene;

b. adding the C₁-C₄ straight or branched chain alcoholic solvents such as methanol, ethanol, isopropanol, n-butanol, iso-butanol and tertiary butanol, preferably methanol to the resulting reaction solution obtained in step (a);

c. distilling off the solvent from the reaction solution of step (b) accompanied by cooling the reaction mass to a temperature of 10-50° C., preferably 25-30° C.; and

d. filtering the compound obtained in step (c) followed by drying the compound at a temperature of 30-100° C. to afford the crystalline Form III of losartan potassium.

The invention is further defined by reference to the following examples describing in detail the preparation of the compound and the compositions of the present invention, as well as their utility. It will be apparent to those skilled in the art, that many modifications, both to materials, and methods, may be practiced without departing from the purpose and interest of this invention.

### REFERENCE EXAMPLE

Preparation of Trityl Losartan

An aqueous solution of sodium hydroxide (15 grams in 700 ml water) was added to a mixture of 2-n-butyl-4-chloro-5-formyl imidazole (54 grams), and tetrabutyl ammonium bromide (14 grams) in toluene (1000 ml), and the resulting reaction mixture was stirred for 20-30 minutes at a temperature of 30-35° C. N-(triphenyl methyl)-5-[4′-(bromomethyl)biphenyl-2-yl]tetrazole) (170 grams) was added to the resulting reaction mixture and stirred for another 33-35 hours. The completion of the reaction was confirmed by Thin Layer Chromatographic (TLC) testing of aliquots withdrawn. The organic layer was separated from the reaction mass and washed with dilute a sodium hydroxide solution (40 ml), and further with water (600 ml). Sodium borohydride (4.0 grams) was added then to the organic layer, and heated to a temperature of 40-45° C., and methanol (40 ml) was added and the temperature maintained for 1½-2 hours. The reaction mass was washed with water (3×200 ml), cooled to a temperature of 0-5° C. and stirred for 1½-2 hours to allow the separation of the solid, which was filtered and dried to afford the desired Trityl Losartan compound (140-145 g).

### Example 1

Trityl losartan (2-n-butyl-4-chloro-1-[(2′-(1-triphenylmethyl-1H-tetrazole-5-yl)-1,1′-biphenyl-4-yl)methyl]1H-imidazole-5-methanol) (125 grams) was prepared as described above in the Reference Example, placed in a mixture of an aqueous solution of potassium hydroxide (11 g in 125 ml water) and methanol (1250 ml), and refluxed until the reaction was substantially complete. The solvent was distilled off the solution under vacuum, and water (375 ml) was added to the residual mass, which was then stirred for 30 minutes, filtered and washed with water (150 ml). The thus obtained filtrate was washed with toluene (2×110 ml), and the aqueous layer was separated from the resulting bi-phasic mixture. Water was distilled off the aqueous layer, and any remaining water traces were removed under reflux as an azeotrope formed by addition of toluene (350 ml). Methanol (100 ml) and carbon (5.5 grams) were added, and the residue stirred for 30 minutes until a clear dissolution was attained. The carbon was filtered off, and washed with methanol (50 ml), and the methanol distilled off, and the reaction mass cooled to a temperature of 20-25° C. to separate the solid mass. The separated solid was filtered, washed with methanol (50 ml) and dried at a temperature of 80-90° C. to obtain crystalline Form-III Losartan Potassium (Weight: 75.0 g; Yield: 86.5

### Example 2

Trityl losartan (2-n-Butyl-4-chloro-1-[(2′-(1-triphenylmethyl-1H-tetrazole-5-yl)-1,1′-biphenyl-4-yl)methyl]1H-imidazole-5-methanol) (125 grams; 0.188 moles) was prepared as described above in the Reference Example, placed in a mixture of an aqueous solution of potassium hydroxide (11 g; 0.196 moles) in water (125 ml)), and methanol (1250 ml), and refluxed until the reaction was substantially complete. The solvent was distilled off the reaction solution under vacuum, and water (325 ml) added to the residual mass, stirred for 30 minutes, the pH adjusted to 8.2-8.8, and the mass filtered. The filtrate was washed with de-mineralized water (150 ml), and the water was distilled off. Any remaining traces of water were removed by formation of an azeotrope with addition of toluene (400 ml) under reflux condition. The resulting residue was dissolved in methanol (80 ml), the solvent distilled off, and the residual mass cooled to a temperature of 5-10° C., filtered, and dried to yield crystalline polymorph Form III of Losartan Potassium (Weight: 43.0 g)

### Example 3

Crystalline Form I of losartan potassium (20.0 grams) was dissolved in toluene (160 ml) at a temperature of 70° C., methanol (30 ml) added to the solution, and stirred for 10 minutes. The methanol solvent was distilled off under vacuum, and the residual mass cooled to a temperature of 25-30° C. and stirred for 15-30 minutes. The solid was filtered, washed with toluene (20 ml) and dried to a temperature of 95-105° C. to yield crystalline Form III of losartan potassium (Weight: 19 grams; Yield: 95%).

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

What is claimed is:

1. A compound that is a crystalline Form III of losartan potassium.

2. The compound of claim 1 having X-ray powder diffraction pattern substantially as shown in FIG. 1.

3. The compound of claim 1 having an X-ray diffraction pattern expressed in terms of 2 theta angles and obtained with a copper K X-radiation source, wherein said X-ray powder diffraction pattern includes five or more peaks selected from the group consisting of peaks with 2 theta angles of 7.15±0.09, 7.58±0.09, 8.04±0.09, 12.38±0.09, 13.23±0.09, 13.91±0.09, 15.27±0.09, 16.04±0.09, 17.19±0.09, 17.79±0.09, 18.48±0.09, 18.76±0.09, 19.29±0.09, 19.57±0.09, 20.73±0.09, 21.58±0.09, 24.19±0.09, 24.90±0.09, 25.67±0.09, 26.09±0.09, 27.77±0.09, 28.91±0.09, 29.47±0.09 and 30.61±0.09 degrees.

4. The compound of claim 1 having an X-ray diffraction pattern expressed in terms of 2 theta angles and obtained with a copper K X-radiation source, wherein said X-ray powder diffraction pattern includes five or more peaks selected from the group consisting of peaks with 2 theta angles of 7.154, 7.583, 8.042, 12.385, 13.233, 13.911, 15.267, 16.043, 17.194, 17.794, 18.483, 18.76, 19.293, 19.571, 20.728, 21.576, 24.192, 24.904, 25.695, 26.088, 27.773, 28.908, 29.474 and 30.614 degrees.

5. The compound of claim 1 having a Differential Scanning Colorimetery (DSC) thermogram exhibiting a significant endo peak at about 264° C.

6. The compound of claim 5 having a characteristic DSC thermogram substantially as shown in FIG. 2.

7. The compound of claim 1 having a characteristic infrared spectrum exhibiting significant bands at about 1580 cm⁻¹, 1460 cm⁻¹, 1422 cm⁻¹, 1358 cm⁻¹, 1257 cm⁻¹, 1112 cm⁻¹, 1075 cm⁻¹, 999 cm⁻¹, 754 cm⁻¹, and 668 cm⁻¹.

8. The compound of claim 7 having the infrared spectrum substantially as depicted in FIG. 3.

1. 9. The compound of claim 1 having a melting range of about 254 to about 260° C.

10. A composition comprising losartan potassium as a solid, wherein at least 80% by weight of said solid losartan potassium is its crystalline Form III.

11. The composition of claim 10, wherein at least 90% by weight of said solid losartan potassium is its crystalline Form III.

12. The composition of claim 10, wherein at least 95% by weight of said solid losartan potassium is its crystalline Form III.

13. The composition of claim 10, wherein at least 99% by weight of said solid losartan potassium is its crystalline Form III.

14. The composition of claim 10, wherein said solid losartan potassium is substantially free of crystalline Forms I and II of losartan potassium.

15. The composition of claim 10, wherein at least 1% of said solid losartan potassium is not its crystalline Form III.

16. The composition of claim 10, wherein at least 5% of said solid Losartan Potassium is not its crystalline Form III.

17. A pharmaceutical or veterinary composition comprising the compound of claim 1 and a pharmaceutically or veterinarily acceptable carrier or diluent.

18. The composition of claim 17, further comprising one or more pharmaceutically acceptable excipients.

19. The composition of claim 18, wherein said pharmaceutical composition is a solid dosage form for oral administration.

20. The composition of claim 19, wherein said solid dosage form is a tablet.

21. The pharmaceutical or veterinary composition of claim 17, wherein the compound of claim 1 is present in the amount of from about 0.01% to about 99.99% by weight.

22. The pharmaceutical or veterinary composition of claim 21, wherein the compound of claim 1 is present in the amount of from about 1% to about 95% by weight.

23. The pharmaceutical or veterinary composition of claim 22, wherein the compound of claim 1 is present in the amount of from about 2% to about 20% by weight.

24. The pharmaceutical or veterinary composition of claim 23, wherein the compound of claim 1 is present in the amount of from about 1% to about 10% by weight.

25. The pharmaceutical or veterinary composition of claim 17, wherein the carrier or diluent is a solid or a liquid.

26. The pharmaceutical or veterinary composition of claim 17, wherein the carrier or diluent is selected from the group consisting of a derivatized cellulosic material, starch, polyhydroxylated alcohol, and mixtures thereof.

27. The pharmaceutical or veterinary composition of claim 17, further comprising an ingredient selected from the group consisting of lubricants, disintegrants, coloring agents, anti-hygroscopic agents, binders, pH adjusting agents, flavoring agents, or aromatic agents.

28. The pharmaceutical or veterinary composition of claim 17, which is in the form of a topical or systemic formulation.

29. The pharmaceutical or veterinary composition of claim 17, which is in the form of an oral, injectable, transdermal, implantable, inhalable, transmucosal, or dermal formulation.

30. The pharmaceutical or veterinary composition of claim 17, which is in the form of powder, tablets, dragees, capsules, oil, cream, solution, emulsion, or suspension.

31. A process for preparing crystalline Form III of losartan potassium, said process comprising:
a) providing a potassium salt of losartan as a solution in a first alcoholic solvent;
b) cooling said solution thereby causing separation of a solid mass;
c) isolating said solid mass which the Form III crystalline Form III of losartan potassium.

32. The process of claim 31, further comprising removing at least a portion of said first alcoholic solvent before said cooling step.

33. The process of claim 31, further comprising reacting trityl losartan with potassium hydroxide to obtain the starting potassium salt of losartan.

34. The process of claim 33, wherein said reacting step includes contacting said trityl losartan with the potassium hydroxide in a second alcoholic solvent and heating said second alcoholic solvent to reflux until the reaction is substantially complete.

35. The process of claim 34, further comprising removing at least a portion of said second alcoholic solvent, and combining the reaction mixture with water and a water-immiscible solvent to form a two-phase liquid system.

36. The process of claim 35, further comprising separating said layers of said two-phase liquid system, isolating the aqueous layer, and reducing the amount of water present therein.

37. The process of claim 36, further comprising combining the reduced aqueous layer with a second water-immiscible solvent capable of forming an azeotropic mixture with water, and heating said second water-immiscible solvent to reflux with removal of the distillate thereby reducing the amount of the water.

38. The process of claim 37, further comprising adding a lower alkanol thereby providing said starting solution of the potassium salt of losartan in the first alcoholic solvent.

39. The process of claim 38, wherein substantially all of said first alcoholic solvent is the lower alkanol.

40. The process of claim 39, wherein the lower alkanol is a C₁-C₄ straight or branched chain alkanol.

41. The process of claim 40, wherein said lower alkanol is selected from the group consisting of methanol, ethanol, isopropanol, n-butanol, iso-butanol, tert-butanol, and mixtures thereof.

42. The process of claim 40, wherein said lower alkanol is methanol.

43. The process of claim 31, wherein said first alcoholic solvent is a mixture of lower alkanol and at least one aromatic solvent.

44. The process of claim 33, wherein the trityl losartan and the potassium hydroxide are reacted at the molar ratio ranging from about 0.5:1.5 to about 1.5:0.5.

45. The process of claim 31, wherein said cooling step is carried out at the temperature ranging from about 0° C. to about 50° C.

46. The process of claim 31, wherein said isolating step is filtration of said solid mass.

47. The process of claim 34, wherein said second alcoholic solvent is different from said first alcoholic solvent.

48. The process of claim 34, wherein said second alcoholic solvent is methanol, ethanol, isopropanol, n-butanol, iso-butanol, tert-butanol, or a mixture thereof.

49. The process of claim 37, wherein said second water-immiscible solvent is different than said first water-immiscible solvent.

50. The process of claim 37, wherein said second water-immiscible solvent and said first water-immiscible solvent, which may be same or different, are selected from the group consisting of benzene, xylene, toluene, ethyl benzene, or mixtures thereof.

51. The process of claim 45, further comprising drying said separated mass at the temperature of from about 30 to about 100° C.

52. The process of claim 43, wherein said at least one aromatic solvent is selected from the group consisting of benzene, xylene, toluene, ethyl benzene, or mixtures thereof.

53. The process of claim 52, wherein said providing step includes dissolving a crystalline Form I of potassium losartan in said at least one aromatic solvent and adding said lower alkanol thereto.

54. The process of claim 53, wherein said lower alkanol is selected from the group consisting of methanol, ethanol, isopropanol, n-butanol, iso-butanol, tert-butanol, and mixtures thereof.

55. The process of claim 53, wherein said lower alkanol is methanol.

56. The process of claim 53, further comprising removing at least a portion of said first alcoholic solvent.

57. The process of claim 56, further comprising cooling the reaction mass to cause separation of a solid mass.

58. The process of claim 57, further comprising isolating the separated mass which is the crystalline Form III of potassium losartan.

59. The process of claim 43, wherein the aromatic solvent comprises toluene.

60. The process of claim 53, wherein the crystalline Form I losartan potassium is combined with said aromatic solvent at a temperature of from about 50° C. to about 80° C.
